(19) United States
(12) Patent Application Publication
(10) Pub. No.: US 2005/0119643 A1
(43) Pub. Date: Jun. 2, 2005

(54) Method and apparatus for opto-thermo-mechanical treatment of biological tissue

(76) Inventors: Emil Naumovich Sobol, Moscow (RU); Viktor Nikolaevich Bagratashvili, Moscow (RU)

Correspondence Address: BURNS DOANE SWECKER & MATHIS L L P, POST OFFICE BOX 1404, ALEXANDRIA, VA, 22313-1404, US

(21) Appl. No.: 10/942,981

(22) Filed: Sep. 17, 2004

(30) Foreign Application Priority Data
Sep. 18, 2003 (RU) 2003128064

(51) Int. Cl. A61B 18/20; A61N 5/06

(52) U.S. Cl. 606/9; 606/10; 607/88; 607/89

(57) ABSTRACT

The invention relates to a method and apparatus for opto-thermo-mechanical treatment of biological tissue. A biological tissue area 8 is irradiated with a radiation in the optical wavelength range with predetermined parameters, the radiation being modulated and spatially formed under a predetermined law; the irradiation is accompanied by simultaneous thermal and mechanical treatment of the area 8; concurrently with the irradiation of the biological tissue area, spatial distribution of physico-chemical and geometrical characteristics is measured both in the zone of direct optical treatment and in close vicinity, using a control diagnostic system 4; a data processing unit 7 coordinates parameters of optical radiation spatial formation and modulation with each other and with the biological tissue characteristics and provides a control signal to an optical radiation power and time modulation control unit 2 and a device 3 for delivering optical radiation and forming spatial distribution of optical radiation power on the surface and in the bulk of the biological tissue 8. Optical radiation parameters are adjusted responsive to control signals of the control-diagnostic system 4 during irradiation as a function of continuously changing characteristics of spatial distribution of physico-chemical and geometrical characteristics both in and beyond the directly treated biological tissue area.

### FIELD OF THE INVENTION

The present invention relates in general to medicine, and more specifically to methods of treatment of biological tissues by locally modifying their structure and physical and chemical characteristics.

Deformation and degeneration of biological tissues may cause a great number of diseases which are mainly treated by surgical methods with inherent problems such as high traumatism, profuse bleeding, pain, need for general anesthesia and long stay at hospital.

### BACKGROUND ART

A method for changing the cartilage shape of the rabbit's ear with the aid of a master form and CO₂ laser radiation was first described in experimental work by E. Helidonis, E. Sobol, G. Kavalos, et. al, American Journal of Otolaryngology, 1993, Vol. 14, No. 6, pp. 410-412. Samples of 0.4 to 1 mm thick cartilaginous tissue having various initial deformations (curved and straight) were isolated from the rabbit ear. The initially curved cartilaginous tissue was straightened manually by forceps using an external mechanical action, and the samples of straight tissue were curved. The samples were then fastened by needles to a wooden master form and irradiated with CO₂ laser in a scanning mode. In this way a stable change was achieved in the shape of cartilaginous tissue isolated in advance, for transplantation into a live body. However, since the tissue should be isolated from the body by making use of an injuring instrument, this method is very traumatic.

Another widely known work (see E. Helidonis, E. Sobol, G. Velegrakis, J. Bizakis, Laser in Medical Science, 1994, Vol. 6, pp. 51-54) describes how the isolated samples of human and rabbit's nasal septum were subjected to deformation with the aid of a master form with subsequent CO₂ laser irradiation. The method produces stable changes in the shape of isolated cartilage, the latter being preserved in physiologic salt solution. This method is applicable in reconstruction operations which are made by isolation of cartilaginous tissue from the patient's body, with its subsequent mechanical and laser treatment and transplantation. Such operations are quite traumatic, labor-consuming and, moreover, they do not rule out the possibility of recurrence of the initial pathology. It should be noted, that the above prior art works deal with the results of in-vitro experiments, for the isolated cartilaginous tissue was subjected to laser irradiation outside the body.

Also known is a method of rhinologic operation for treatment of the cartilage shape of human nasal septum (see Patent RU No. 2114569 of Sep. 7, 1993). An example of clinical application of the method describes straightening of curved human nasal septum with the help of CO₂ laser.

According to the method, the mucous membrane is separated from the curve of the nasal septum, then the cartilaginous plate is straightened and kept in such state with the aid of conventional forceps. The forceps are usually double-branch holding forceps with flat, solid branches which make it possible to grip, bend the cartilaginous plate to the side opposite to the pathologic deformation, and held it throughout the time of irradiation. Then the cartilage is subjected to irradiation along the bend line with a scanning CO₂ laser beam at a speed of 0.03 cm per sec. After irradiation the forceps are removed and the changed form of the nasal septum is visually inspected.

Although stable results have been obtained in clinical tests, applicability of the method in medical practice is highly problematic. The method lacks any control over the cartilage irradiation process. The used radiation penetrates into the cartilage to a depth less than 50 μm, which leads to unavoidable overheating of the surface layer and destruction of perichondrium. In the example of the method's clinical application the mucous membrane and perichondrium are separated, this in itself causing the patient's profuse bleeding and suffering which may eventually contribute to development of atrophic processes.

A method is also known for changing the cartilage form of the dog's tracheal ring (Shapshay S. M., Pankratov M. M. et al, Otol. Rhinol Laryngol, 1996. Vol. 105, pp. 176-181), using laser radiation. In the method, in the event of throat and trachea stenosis, the contracted cartilaginous element of the trachea is cut with the aid of an endoscope and CO₂ laser in order to improve breathing, and next, with the aid of the same endoscope the deformed cartilaginous tissue is irradiated with 1.44 μm Nd:YAG laser beam through the mucous membrane, along the internal surface of the contracted cartilaginous element of the trachea.

The method has the advantage of radiation delivery and visual control of the zone of treatment, especially when modifying the shape of cartilages that are difficult to locate. However, the method is technically complicated and requires consecutive use of two laser treatment sessions. Moreover, to transfer the pathologically deformed section of the cartilaginous tissue to a normal position a considerable external mechanical effort is required. This is done with the aid of a flexible endoscope acting as a mechanical bougie. The endoscope must have sufficient mechanical strength and rigidity. However, due to the limited mechanical strength of the endoscope, the method is applicable solely for broadening the cartilaginous elements with a relatively small radial deformation, some 1-2 mm, no more.

Moreover, the cartilage may be irradiated only over the internal surface of the ring element, with the external side being inaccessible for radiation.

Most closely related to the present invention are a method and apparatus for treatment of deformed cartilaginous tissue, disclosed in International Application WO 01/22863 A2, of Apr. 5, 2001, IPC A61B, Sobol et al.

The method relies on the laser treatment with simultaneous monitoring of the cartilaginous tissue characteristics and modification of laser radiation energy parameters.

A problem with the method is that the advantageous modification of the cartilaginous tissue shape can be attained in a narrow range of laser parameters, while going beyond the range results in tissue injuries or deformation relapse; the control system used in the method relies on measuring the integral characteristic of the biologic tissue without accounting for spatial heterogeneity of the characteristics, which may give rise to erroneous selection of the instant of laser exposure termination. An essential drawback of the method is the lack of control over the characteristics of biologic tissue in the area adjacent to the region directly exposed to laser radiation, so the conditions are created for undesirable effect on surrounding tissues, and the side effect risks are increased. The method is unsuitable for treating injured biologic tissues, such as articular cartilages and intervertebral discs.

A method is known for treating pathologies of intervertebral discs by laser ablation (evaporation) of diskal hernia and decompression. (See D. Choy D S J, Case P B, Fielding W. Percutaneous laser nucleolysis of lumbar disc. New England Journal of Medicine, 1987, 317:771-772) and (See, Daniel S. J. Choy, MD, Peter W. Ascher, MD, and others Percutaneous Laser Disc Decompression, A New Therapeutic Modality, Spine, Volume 17, Number 8, 1992).

The method, however, suffers from the problems of unavoidable overheating of tissues joining the ablation zone and undesirable effect on surrounding tissues, which manifest themselves in scarring, and of high probability of relapses caused by the fact that the method, like a traditional surgical herniotomy, fails to eliminate the fibrous ring defect which is the main cause of the disease.

Therefore, there is no effective and safe approach so far to non-traumatic treatment of diseases caused by deformation and injury of biological tissues.

### SUMMARY OF THE INVENTION

The foregoing problems of the prior art are overcome by the present invention which provides a method and apparatus for opto-thermo-mechanical treatment of biological tissue. The method and apparatus produce controlled spatial and time heterogeneities of temperature and mechanical stress in biological tissues by subjecting the tissues to optical radiation modulated in space and time.

Space and time modulation (STM) of optical radiation is modification of spatial distribution of the radiation power in time, controlled under a predetermined law. The STM involves the pulse periodic nature of laser radiation and known laws of laser beam scanning, but the difference is that the STM provides an arbitrarily specified space and time distribution of optical radiation, and, respectively, it allows modification of laser heating space and time characteristics and thermal stress fields under a predetermined law, i.e. provides more extended opportunities for opto-thermo-mechanical treatment of biological tissues, particularly for controlling temperature and mechanical stress gradients. Recent researches have shown that chondrocytes, fibroblasts and some other cells of biological tissues are sensitive to external mechanical stress fields, specifically the reproductive and regenerative abilities of the cells can be increased or decreased depending on parameters of external mechanical action. No method exists so far for controlled local thermal and mechanical influence upon cells in-vivo. Controllability is required to provide efficiency and predictability of the influence results. Locality is required to prevent the undesirable influence upon surrounding tissues, hence to provide safety of the procedure.

It should be also noted that the method and apparatus in accordance with the invention provide formation of controlled, coordinated space and time heterogeneities in temperature and thermomechanical stress, and acoustic waves in biological tissues.

Local thermal effect on a biologic tissue is necessary to provide local irreversible alteration in microstructure (“local fusion of individual structure elements”) of the biologic tissue, which causes relaxation of mechanical stresses and creation of optimal heterogeneities of residual stresses in the tissue. In accordance with the method of the invention, mechanical influence is exerted upon the tissue, in particular upon biologic cells which participate in tissue regeneration processes; in addition, the controlled thermal effect accelerates all physical and chemical processes underlying the treatment. However, overheating of the tissue causes its denaturation and destruction in the region of direct effect and undesired effects beyond the region (violation of the locality and safety principles).

Long-time effect of biologic tissue treatment depends both from kinetics, degree of completion of irreversible processes, and distribution of residual stresses after termination of laser treatment. As the residual stress field influences the tissue regeneration effect of cells, thermal and mechanical treatment of the biological tissue must be coordinated to achieve positive result (efficiency) and provide safe procedures.

The object of the present invention is to provide a method and apparatus for opto-thermo-mechanical treatment of biological tissue, which ensure efficient and safe approach to non-traumatic treatment of diseases associated with deformations and injuries of biological tissues, by producing controlled residual stresses and controlled spatial distribution of irreversible alterations in the biological tissue structure.

The object is achieved in a method for opto-thermo-mechanical treatment of biological tissue in accordance with the invention, said method comprising:
determining, on the basis of patient's preoperative examination, spatial distribution of physico-chemical and geometrical characteristics of the biologic tissue in an area to be subjected to opto-thermo-mechanical treatment;
if necessary, exerting mechanical action on the biologic tissue area to be treated, in particular, by giving a predetermined shape to the area;
irradiating the biological tissue area by a radiation in the optical wavelength range with predetermined parameters, said radiation being modulated and spatially formed under a predetermined law, with simultaneous thermal and mechanical treatment of said area;
concurrently with said irradiation of the biological tissue area, measuring spatial distribution of physico-chemical and geometrical characteristics both in the zone of direct optical treatment and beyond the area;
coordinating the parameters of spatial formation and modulation of optical radiation with each other and with said biological tissue characteristics;
determining modification of said biological tissue characteristics with respect to the measurements of the characteristics at the preoperative examination step;
adjusting the optical radiation parameters in the course of irradiation responsive to continuously measured characteristics of spatial distribution of physico-chemical and geometrical characteristics both in and beyond the directly treated biological tissue area;
terminating said irradiation of the biological tissue area when desired characteristics of spatial distribution of physico-chemical and geometrical characteristics are obtained, the parameters of opto-thermo-mechanical treatment of the biological tissue being specified such that to provide controlled residual mechanical stress and controlled irreversible modification in the biological tissue structure.

The radiation in the optical wavelength range is laser radiation in the wavelength range of from 0.1 to 11 micrometers.

The laser radiation can be pulsed or continuous.

The laser radiation has a power density in the range of from 1 to 1000 W/cm².

Duration of the irradiation of the biological tissue area by the laser radiation is in the range of from 0.1 sec to 30 min.

The spatial formation of optical radiation, such as laser radiation, comprises:
(a) forming a predetermined distribution of radiation power density on the surface and in the bulk of the biological tissue area;
(b) scanning by laser beam along three coordinates under a predetermined law;
(c) combining steps (a) and (b).

The optical radiation parameters adjusted in the process of irradiation of the biological tissue area responsive to continuously measured characteristics of spatial distribution of physico-chemical and geometrical characteristics, both in and beyond the directly treated biological tissue area, include: radiation wavelength, radiation power, radiation power density and spatial and time law of its modification, and laser radiation modulation and spatial formation parameters, such as modulation percentage and frequency on the surface and in the bulk of the biological tissue, and spatial distribution of radiation power.

The modulation percentage is between 0.1 and 100%, and the modulation frequency is between 0.1 and 10⁹ Hz.

The measurement of spatial distribution of physico-chemical and geometrical characteristics both in and beyond the zone of direct laser treatment is performed with account for spectral content of biological tissue area response to the modulated laser irradiation of said area.

The method in accordance with the invention further comprises measuring oscillation amplitude and phase of the biological tissue area response to the modulated laser irradiation of said area.

The predetermined laser radiation modulation frequency is selected in coordination with resonance frequencies of mechanical oscillations in the biological tissue treatment area.

If necessary, parts of biological tissue, such as skin or mucous membrane covering the biological tissue area to be treated, are locally pressed on prior the irradiating of the biological tissue.

In a second aspect of the present invention an apparatus is provided for treatment of biological tissue, the apparatus comprising: an optical radiation source having an optical radiation power and time modulation control unit optically coupled to a device for delivering optical radiation and forming spatial distribution of optical radiation power density on the surface and in the bulk of the biological tissue, and a control-diagnostic system for determining spatial distribution of physico-chemical and geometrical properties of the biological tissue area to be treated and adjacent area, said control-diagnostic system being connected to the optical radiation source, the optical radiation power and time modulation control unit, and the device for delivering optical radiation and forming spatial distribution of optical radiation power density on the surface and in the bulk of the biological tissue, respectively.

The optical radiation source is a laser radiation source.

The laser radiation source emits laser radiation within the range of from 0.1 to 11 micrometers.

The control-diagnostic system comprises at least one biological tissue state sensor to measure characteristics of the biological tissue area in the treatment region and in close proximity, the sensor being connected to a data processing unit for generating control signals to adjust optical radiation parameters in the irradiation process, and an information visualization and display device.

The at least one biological tissue state sensor in the control- diagnostic system measures physico-chemical and geometrical characteristics of the biological tissue area, such as biological tissue temperature and water concentration, mechanical stresses, light scattering characteristics, velocity of sound, opto-acoustic wave damping factor, and geometrical dimensions of the biological tissue.

Responsive to signals received from the at least one biological tissue state sensor, the signal processing unit of the control-diagnostic system provides signals to the optical radiation source, the optical radiation and time modulation control unit, the device for delivering optical radiation and forming spatial distribution of optical radiation power density on the surface and in the bulk of the biological tissue, respectively.

The optical radiation and time modulation control unit is an electro-optical modulator, or acousto-optical modulator, or mechanical modulator.

Furthermore, the optical radiation is modulated by modifying the pumping power, e.g. of the laser radiation source.

The device for delivering optical radiation and forming spatial distribution of optical radiation power density on the surface and in the bulk of the biological tissue includes, optically coupled, a forming optical system and an electro-optical scanner.

The device for delivering optical radiation and forming spatial distribution of optical radiation power density on the surface and in the bulk of the biological tissue includes, optically coupled, a forming optical system and a raster system.

Furthermore, the forming optical system is a length of optical fiber, or a lens-and-mirror system adapted to deliver laser radiation from the optical radiation source to the biological tissue area.

The information visualization and display device in accordance with the invention includes e.g. an endoscope and a display to output image of the biological tissue area, or an optical coherent tomograph.

The information visualization and display system measures geometrical characteristics of the biological tissue area.

The control-diagnostic system provides feedback on the basis of opto-thermal response of the biological tissue to the time-modulated laser radiation.

Feedback is provided by the control-diagnostic system on the basis of analysis of spectral content of the biological tissue response to the modulated laser radiation.

Feedback is provided by the control-diagnostic system on the basis of analysis of amplitude and phase of the biological tissue response to the modulated laser radiation.

Time law of the laser radiation modulation, in particular modulation amplitude, depth, frequency and shape are determined by the control-diagnostic system from preoperative examination data and updated during laser treatment responsive to control signal from the control-diagnostic system.

Formation law of the laser radiation spatial distribution is determined from preoperative examination data and updated during laser treatment responsive to control signal from the control-diagnostic system.

Parameters of laser radiation scanning or spatial distribution are determined from preoperative examination data and updated during laser treatment responsive to control signal from the control-diagnostic system.

In the apparatus, the laws of laser radiation modulation and spatial formation are coordinated on the basis of preoperative examination data and updated during laser exposure responsive to control signal from the control-diagnostic system.

Feedback is further provided on the basis of opto-acoustic response of the biological tissue to the modulated laser radiation formed with a predetermined spatial distribution on the surface and in the bulk of the biological tissue.

Feedback is further provided on the basis of opto-electrical response of the biological tissue to the modulated laser radiation formed with a predetermined spatial distribution on the surface and in the bulk of the biological tissue.

Feedback is further provided on the basis of monitoring the changes in biological tissue optical properties under laser radiation modulated and formed with a predetermined spatial distribution on the surface and in the bulk of the biological tissue.

In the apparatus in accordance with the present invention, the at least one biological tissue state sensor of the control-diagnostic system can be positioned directly in the biological tissue area with the aid of a surgical instrument.

The method and apparatus in accordance with the present invention offer the following advantages:
(1) Reduced temperature at which medical effect is achieved, extended range of permissible treatment regimes, widened sphere of safe application of the method in medicine (in particular, for treatment of spine pathologies);
(2) Optimized (enhanced) opto-thermo-mechanical effect on biological tissues, in particular owing to (mechanical and acoustic) oscillation effects and occurring resonances;
(3) Improved accuracy and safety of the feedback system operation;
(4) Eliminated undesirable effect on surrounding tissue and reduced or completely eliminated probability of complications and undesirable side effects.

Of importance is the fact that the use of laser radiation modulation allows the operation of control-diagnostic systems to be fundamentally modified such that the system can record the biological tissue response precisely to the modulated radiation. This allows recording of the opto-thermo-mechanical response, and analysis of the response spectral content and phase, not only the signal amplitude as in Application WO 01/22863 A2.

### BRIEF DESCRIPTION OF THE DRAWINGS

The invention will be described below with reference to its exemplary embodiments and the attached drawing wherein:

FIG. 1 shows a structural diagram of an apparatus for treatment of biological tissue, suitable for implementing a method of opto-thermo-mechanical treatment of biological tissue.

### DESCRIPTION OF THE PREFERRED EMBODIMENT

A method of opto-thermo-mechanical treatment of biological tissue, which is also the subject of the present invention, will be described below as implemented by an apparatus in accordance with the invention.

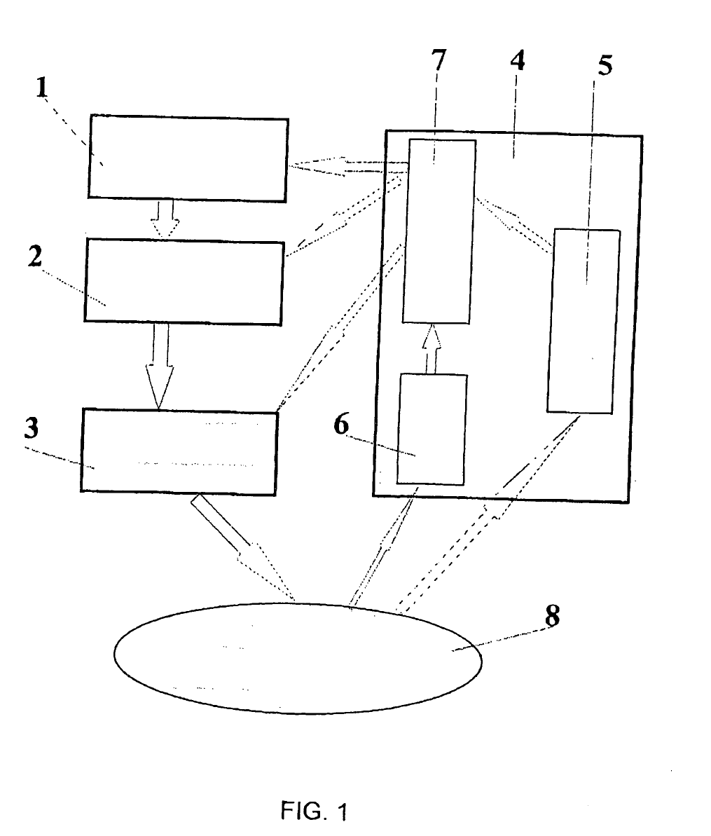

The method and apparatus will be described with reference to FIG. 1. An apparatus for treatment of biological tissue shown at FIG. 1 comprises an optical radiation source 1; an optical radiation power and time modulation control unit 2; a device 3 for delivering optical radiation and forming spatial distribution of optical radiation power density on the surface and in the bulk of the biological tissue; a control-diagnostic system 4 including an information visualization and display device 5, at least one biological tissue state sensor 6 and a data processing unit 7; reference numeral 8 denotes the biological tissue area to be treated.

The optical radiation source 1 is a laser radiation source which can be pulsed-periodic, or continuous with time-modulated output power. This may be e.g. pulse-periodic Nd:YAG laser emitting at 1.32 μm wavelength or continuous fiber laser with periodically modulated emission at 1.56 μm wavelength.

The optical radiation power and time modulation control unit 2 may be integrated in the laser excitation system, or an external unit not connected directly to the laser. In the first case, radiation can be modulated by modulating the laser pump power, e.g. by supply voltage. In the second case, radiation can be modulated e.g. by an electro-optical modulator, an acousto-optical modulator or a mechanical modulator (circuit breaker).

The device 3 for delivering optical radiation and forming spatial distribution of optical radiation power density on the surface and in the bulk of the biological tissue can be of two types. In the first type, periodic or aperiodic scanning over the biological tissue by laser beam along three coordinates is used. Scanning frequency and amplitude, as well as the laser spot size can be varied so that to provide optimal conditions of tissue treatment. The scanning device can be e.g. an electro-optical scanning unit.

In the second type, an optical (e.g. raster) system generates a laser spot on the biological tissue surface with a predetermined, in particular space-modulated, radiation (e.g. periodically changing in space) with power density distribution over the spot. The laser radiation is delivered from the radiation source 1 to the biological tissue by the forming optical system comprising a lens-and-mirror system or a length of optical fiber.

The control-diagnostic system 4 comprises an information visualization and display device 5 such as endoscope with a display or an optical coherent tomograph, at least one biological tissue state sensor 6 and a data processing unit 7 which generates from output of the least one biological tissue state sensor control instructions for the optical radiation source 1, the optical radiation power and time modulation control unit 2, and the device 3 for delivering optical radiation and forming spatial distribution of optical radiation power density.

The biological tissue state sensor(s) 6 is a device which records changes in physico-chemical characteristics of the biological tissue exposed to opto-thermo-mechanical treatment, and depending on the type of treatment, position and size of the target biological tissue; the device can comprise dedicated temperature sensors; acoustic signal amplitude, phase and frequency sensors; mechanical stress sensors; scattered light amplitude, phase, frequency and spatial distribution sensors and sensors of water concentration in irradiated biological tissue.

The data processing unit 7 can be at least one computer board, such as Intel Pentium-2 processor, DC-XG Legacy Sound System card or virtual multi-channel oscillograph integrated in personal computer to process signals received from the sensors 6 of the control-diagnostic system and generate, under a predetermined algorithm, control signals for the optical radiation source 1, the optical radiation power and time modulation control unit 2, and the device 3 for delivering optical radiation and forming spatial distribution of power density responsive to changes in radiation power, modulation parameters and spatial distribution of power density on the surface and in the bulk of the biological tissue, or for switching off the laser.

Radiation from the optical radiation source 1 is time-modulated on the basis of preoperative examination data by the optical radiation power and time modulation control unit 2, and formed and delivered to the irradiated biological tissue by the device 3 for delivering optical radiation and forming spatial distribution of optical radiation power density. The at least one biological tissue state sensor 6 is fixed in close proximity to or in direct contact with the exposed tissue so that to optimally get information about biological tissue state.

A method of opto-thermo-mechanical treatment of biological tissue in accordance with the invention is accomplished in the following manner. A biological tissue area to be treated is located e.g. by an information visualization and display device 5 or on the basis of patient's preoperative tomography examination data. Then the biological tissue state sensor(s) 6 is mounted, the control-diagnostic system 4 is enabled, and spatial distribution of physico-chemical and geometrical characteristics of the biological tissue in the target area is determined, e.g. by measuring spatial distribution of mechanical stress by a microtensometer, measuring acoustic oscillation damping factor at excitation of opto-acoustic waves by low-intensity modulated laser emission (power density of 0.01-0.5 W/cm²) at which temperature variation in the laser exposure zone does not exceed 1 K. Spatial distribution of temperature in the biologic tissue is measured e.g. by a microthermocouple or a scanning infrared imager. Geometrical characteristics (shape and dimensions) of the target biological tissue area are determined by the information visualization and display device 5, such as an optical coherent tomograph. Spatial distribution of biological tissue structure heterogeneities is determined e.g. by an optical coherent tomograph.

Then the patient's preoperative examination data is processed by the data processing device 7 which outputs, under a predetermined algorithm, recommendations for selection of initial laser radiation parameters. In particular, the laser spot shape and dimensions and the scanning law are chosen in accordance with geometrical characteristics and spatial distribution of stresses in the biological tissue area to be treated. A predetermined optical radiation modulation frequency is selected e.g. so that to match mechanical oscillation resonance frequencies in the treated biological tissue area. Initial parameters of laser radiation are specified, e.g. wavelength 1.5 μm, laser source power 2 W, laser radiation spot shape, e.g. circle of 1 mm diameter, modulation frequency 26 Hz, modulation percentage 80%, and the law of radiation scanning in space (along three coordinates) and time.

When a deformed cartilaginous tissue is treated, a predetermined shape is given, if necessary, to the target biological tissue area by mechanical action with the aid of a surgical instrument.

A mechanical instrument can be also used, if necessary, to locally press on biological tissue parts, e.g. skin or mucous membrane, which cover the biological tissue area to be treated. The local pressure enhances safety of opto-thermo-mechanical treatment. It locally decreases water concentration and, respectively, locally reduces the radiation absorption coefficient in near-surface layers of the biological tissues, this offsetting the temperature maximum into the bulk of the target biological tissue and preventing overheating and injury of surface layers, such as skin, mucous membrane and perichondrium.

An apparatus for opto-thermo-mechanical treatment of biological tissue in accordance with the invention operates in the following manner.

Optical, e.g. laser radiation from a radiation source 1 is time-modulated by an optical radiation power and time modulation control unit 2 (e.g. acousto-optical modulator) and is delivered by an optical forming system, e.g. optical fiber, to a device 3 for forming spatial distribution of optical radiation power density, e.g. an optical microlens raster located near the surface (at 5-10 mm distance) of the target biological tissue area. Heating of the biological tissue by the laser radiation causes modification of spatial distribution of geometrical and physico-chemical characteristics thereof, e.g. temperature field, stress field or laser light scattering diagram, which are continuously monitored by a visualization and display device 5, e.g. an optical coherent tomograph, such as 1MALUX, sensors 6, such as a scanning IR radiometer or strain microsensor based on resistive-strain sensor, or an optical multichannel analyzer (OMA), such as MOPC-11. Signals from the sensors 6 and the information visualization and display device 5 are continuously provided to the data processing unit 7 where they are processed and output to a video display to enable continuous visual monitoring of the exposed tissue characteristics and manual control of radiation parameters. At the same time, the data processing unit 7 generates instructions under a predetermined algorithm responsive to signals from the sensors 6 and the information visualization and display device 5 for the optical radiation power and time modulation control unit 2 and the device 3 for delivering optical radiation and forming spatial distribution, to modify power, parameters of time modulation and spatial distribution of optical radiation power density, and a disable command to switch off the optical radiation source 1 when required characteristics of the exposed tissue are obtained, e.g. when temperature of nasal septum is 70° C.

A method of opto-thermo-mechanical treatment of biological tissue will be further described with reference to the following examples.

### EXAMPLE 1

A 49-year old man applied to the clinic with complaints of pain in the lumbar spine after a year of intervertebral disk herniotomy. Preoperative examination, including computer tomography and discography, revealed spine instability and defect of fibrous ring of the operated intervertebral disk.

To treat the pathology, the defect topology and dimensions were first determined, and distribution of mechanical stresses in the fibrous ring area was defined by a microtensometer introduced into the intervertebral disk through a needle of 1.6 mm diameter. The laser radiation source was Er-glass fiber laser emitting at 1.56 μm wavelength with radiation power between 0.2 and 5 W, radiation modulation frequency in the range of from 1 to 80 Hz and percentage from 50 to 100%. Based on preoperative examination, the following laser radiation initial parameters were chosen: laser source power 0.9 W; modulation frequency 5 Hz, modulation percentage 80%. Local anesthesia by Novocain injection was applied. The radiation was delivered to the defect zone through a fiber waveguide of 600 μm diameter inserted into a metal needle 25 cm long with 1.2 mm external diameter.

The control-diagnostic system included two sensors: an acoustic sensor for measuring biological tissue opto-acoustic response to the modulated laser exposure and a microthermocouple for measuring temperature. Both sensors were attached to a second metal needle 25 cm long with 2 mm diameter, which was introduced into the intervertebral disk at an angle of 30 degrees to the first needle and moved in the course of exposure to a new position every 5 seconds with 0.5 mm steps. Endoscope system was used to visualize position of the two needles and the treated zone. Optical coherent tomograph was used to record modifications in the fibrous ring tissue. Total treatment time was 160 seconds. Temperature measurements showed that the temperature increase in the fibrous ring near the spinal channel was no more than 1.2° C., so the opto-thermal-mechanical treatment was safe. Spinal pain significantly reduced immediately after the procedure. Control examination by tomography, diskography and measurement of acoustic wave distribution velocity after 3 and 9 months of the treatment demonstrated that the fibrous ring defect was healed with grown cartilaginous tissue. Thus, the proper selection of opto-thermo-mechanical treatment of damaged fibrous ring of intervertebral disk by modulated laser exposure provided the process of controlled irreversible modification of the ring structure and resulted in stable medical effect—removal of pain and spine instability.

### EXAMPLE 2

A 55-year-old woman applied to the clinic for the reason of aesthetic defect of the shape of nose. Preoperational examination by a visualization and display device including endoscope and optical coherent tomograph showed bend of cartilage plates of the nose halves without nasal bone disorders.

A laser radiation source was Nd:YAG solid-state pulse-periodic laser emitting at 1.32 μm wavelength with average radiation power from 0.3 to 5 W, pulse duration 1 ms, pulse repetition rate from 10 to 700 Hz. A radiation spatial distribution unit provided radiation focusing in the form of four round spots 0.4 to 3 mm in diameter, spaced at 0.5 to 10 mm, and scanning the radiation along three coordinates with a velocity from 0.1 to 20 cm/s.

Used as feedback was a scattered light phase obtained by exposing the nose halves to a supplementary low-intensity light source—0.68 μm diode laser, and a signal of microthermocouple. Two symmetrical cartilages of nose halves were given a predetermined shape by a surgical instrument that provided smooth curvature to cartilages inside the nose halves without surgical isolation thereof. The two cartilage plates were alternatively exposed to laser radiation pulses with repletion rate of 20 Hz via an optical fiber and a raster optical system. Laser radiation power was 2.5 W during first 12 seconds, and after receiving a microthermocouple signal indicative of temperature stabilization at 52° C. in the cartilage being heated, the radiation power was increased up to 4.4 W. The laser was switched off after receiving a signal of light scattering signal 180° phase rotation from the control-diagnostic system, which indicated that the process of stress relaxation in the heated cartilage was over. Heating time for two individual cartilage plates at 4.4 W laser power was 4.2 and 5.1 sec, respectively, at the same achieved 68° C. temperature. Postoperative examination by an optical coherent tomograph immediately after operation and after 6 months demonstrated that the newly made configuration of both nose halves was stable without any visible damage to mucous membrane and other adjacent tissues. Thus, the selected conditions of opto-thermo-mechanical treatment by time-modulated and spatially-formed laser exposure of deformed cartilage plates of nose halves provided a process of controlled irreversible modification of the nose structure and, as consequence, resulted in the desired cosmetology effect—recovery of the specified shape of deformed nose halves.

### EXAMPLE 3

A 13-years old boy applied to the clinic with complaints of a difficulty in nasal respiration. Preoperative examination by both an endoscope imaging system and an optical coherent tomograph showed the bend in the nasal septum cartilage section associated with nasal trauma, without pathological deformation of bone tissue.

A laser radiation source was Er-glass fiber laser emitting at 1.56 μm wavelength with radiation power from 0.2 to 5 W, initial radiation modulation with frequency 365 Hz and percentage 30%.

A control-diagnostic system comprising an opto-acoustic sensor and a microtensometer was used. The laser source was switched on at a reduced power level of 0.1 W, and a spot of 1 mm diameter linearly scanned the target cartilage tissue area with 0.1 Hz frequency and 5 cm amplitude; spatial distribution of opto-acoustic signal amplitude was measured so that a data processing device (reference numeral 7 at FIG. 1) could select initial laser power spatial distribution. As the result, the laser spot on the mucous membrane surface through which the cartilages were irradiated, was selected to have the shape of a line 29 mm long and 0.3 mm wide, positioned along the cartilage plate bend line at 5 mm distance from the cartilage growth zone, this preventing its overheating. Straightening and fixation of a predetermined nasal septum shape, and mechanical pressure on the mucous membrane covering the cartilaginous tissue in the treated area were performed by a surgical instrument. Laser heating was conducted at 4.5 W laser radiation power during 6 sec. The laser was switched off after receiving a microtensometer signal indicating that 10% spatial heterogeneity of residual stresses was achieved in the nasal septum. The typical step of heterogeneities was 300 μm which correlates with typical distance between cartilaginous tissue active cells—chondrocytes. During the operation made under the application anesthesia, the patient experienced no pain and left clinic on his own in 30 minutes after the operation end. Tomographic and rhinoscopic examination conducted immediately after exposure and after 3 and 9 months revealed that the newly given shape of the nasal septum cartilage was stable with equal gas flows through both nasal passages. Optical coherent tomography revealed no damages of mucous membrane joining the nasal septum, and perichondrium. Consequently, the selected conditions of opto-thermo-mechanical treatment by time-modulated and spatially-formed laser exposure of deformed nasal septum cartilage provided controlled heterogeneity of residual stresses in the cartilage, which resulted in the desired medical effect—straightening the nasal septum and recovery of normal respiration. In addition, the cartilage shape recovery procedure was safe, because in the process of laser opto-thermo-mechanical treatment the cartilage growth zones stayed untouched, this preventing abnormal development and disproportions occurring after traditional highly traumatic surgical treatment.

The present invention provides a novel method of controlled opto-thermo-mechanical impact on spatial heterogeneity of temperature, stresses and structure of biological tissues. The method and apparatus for opto-thermo-mechanical treatment of biological tissue can be used in different medical spheres, in particular in otolaryngology and cosmetology—for correction of cartilage shape; in ophthalmology—for correction of the cornea shape; orthopedic and spinal surgery—for treatment of joint and intervertebral disk pathologies.

### LIST OF REFERENCE NUMERALS IN THE FIGURE

1—laser radiation source

2—radiation parameter and modulation control unit

3—radiation delivery and spatial distribution formation unit

4—control-diagnostic system

5—information visualization and display device

6—biological tissue state sensor(s)

7—data processing unit

8—biological tissue area to treated

1. A method for opto-thermo-mechanical treatment of biological tissue, comprising the steps of:
determining, on the basis of a patient's preoperative examination, a spatial distribution of physico-chemical and geometrical characteristics of the biologic tissue in an area to be subjected to the opto-thermo-mechanical treatment;
if necessary, giving a predetermined shape to the biological tissue area to be treated by exerting a mechanical action thereon;
irradiating the biological tissue area by a radiation in an optical wavelength range with predetermined parameters, said radiation being modulated and spatially formed under a predetermined law, with a simultaneous thermal and mechanical treatment of said area;
concurrently with said irradiation of the biological tissue area, measuring the spatial distribution of physico-chemical and geometrical characteristics both in a zone of a direct optical exposure and in a close vicinity of said area;
coordinating parameters of an optical radiation spatial formation and modulation with each other and with said biological tissue characteristics;
determining modification of said characteristics with respect to the measurements of the characteristics at the preoperative examination step;
adjusting the optical radiation parameters in a course of irradiation responsive to continuously measured characteristics of the spatial distribution of physico-chemical and geometrical characteristics both in the directly treated biological tissue area and in the close vicinity of said area;
terminating said irradiating of the biological tissue area when a desired characteristics of the spatial distribution of physico-chemical and geometrical characteristics are obtained, parameters of the opto-thermo-mechanical treatment of the biological tissue being specified such that to provide a controlled residual mechanical stress and a controlled irreversible modification of the biological tissue structure.

2. The method as set forth in claim 1, wherein said radiation in the optical wavelength range is a laser radiation in a range from 0.1 to 11 micrometers.

3. The method as set forth in claim 2, wherein said laser radiation is a pulsed or continuous radiation.

4. The method as set forth in claim 2, wherein said laser radiation has a power density in a range from 1 to 1000 W/cm².

5. The method as set forth in claim 1 wherein a duration of said irradiation of the biological tissue area by the optical radiation, such a laser radiation is selected from a range from 0.1 sec to 30 min.

6. The method as set forth in claim 1, wherein said spatial formation of the optical radiation, such as a laser radiation, comprises:
(a) forming a predetermined distribution of a radiation power density on a surface and in a bulk of the biological tissue area;
(b) scanning by a laser beam along three coordinates under a predetermined law;
(c) combining steps (a) and (b).

7. The method as set forth in claim 1, wherein said optical radiation parameters adjusted in the process of irradiation of the biological tissue area responsive to the continuously measured characteristics of the spatial distribution of physico-chemical and geometrical characteristics, both in and beyond the directly treated biological tissue area, include: a radiation wavelength, a radiation power, a radiation power density and a spatial and time law of its modification, and a laser radiation modulation and spatial formation parameters, such as a modulation percentage and a frequency on the surface and in the bulk of the biological tissue, and spatial distribution of radiation power.

8. The method as set forth in claim 7, wherein said modulation percentage is between 0.1 and 100%, and the modulation frequency is between 0.1 and 10⁹ Hz.

9. The method as set forth in anyone of claim 2, wherein said measuring of the spatial distribution of physico-chemical and geometrical characteristics both in and beyond the zone of the direct laser treatment is performed with account for a spectral content of the biological tissue area response to a modulated laser irradiation of said area.

10. The method as set forth in claim 9, further comprising measuring an oscillation amplitude and a phase of the biological tissue area response to the modulated laser irradiation of said area.

11. The method as set forth in 8, wherein said predetermined laser radiation modulation frequency is selected in coordination with resonance frequencies of mechanical oscillations in the biological tissue treatment area.

12. The method as set forth in claim 1, wherein, if necessary, parts of the biological tissue, such as a skin or a mucous membrane covering the biological tissue area to be treated, are locally pressed on prior to said irradiating of the biological tissue.

13. An apparatus for treatment of biological tissue, comprising: an optical radiation source having an optical radiation power and a time modulation control unit optically coupled to a device for delivering optical radiation and forming a spatial distribution of the optical radiation power density on the surface and in the bulk of the biological tissue area, and a control-diagnostic system for determining spatial distribution of a physico-chemical and geometrical characteristics of the biological tissue area to be treated and adjacent area, said control-diagnostic system being connected to the optical radiation source, the optical radiation power and the time modulation control unit, and the device for delivering optical radiation and forming spatial distribution of optical radiation power density on the surface and in the bulk of the biological tissue, respectively.

14. The apparatus as set forth in claim 13, wherein said optical radiation source is a laser radiation source.

15. The apparatus as set forth in claim 14, wherein said laser radiation source emits the laser radiation in a range from 0.1 to 11 micrometers.

16. The apparatus as set forth in claim 13, wherein the control-diagnostic system comprises at least one biological tissue state sensor to measure characteristics of the biological tissue area in the treatment region and in close proximity, the sensor being connected to a data processing unit for generating control signals to adjust the optical radiation parameters in the irradiation process, and an information visualization and display device.

17. The apparatus as set forth in claim 16, wherein said at least one biological tissue state sensor in the control-diagnostic system measures physico-chemical and geometrical characteristics of the biological tissue area, such as a biological tissue temperature and water concentration, mechanical stresses, light scattering characteristics, velocity of sound, opto-acoustic wave damping factor, and geometrical dimensions of the biological tissue.

18. The apparatus as set forth in claim 16, wherein the signal processing unit of the control-diagnostic system, responsive to signals received from said at least one biological tissue state sensor, provides control signals to the optical radiation source, the optical radiation power and time modulation control unit, the device for delivering optical radiation and forming spatial distribution of the optical radiation power density on the surface and in the bulk of the biological tissue, respectively.

19. The apparatus as set forth in claim 13, wherein said optical radiation power and time modulation control unit is an electro-optical modulator, or acousto-optical modulator, or mechanical modulator.

20. The apparatus as set forth in claim 13, wherein said optical radiation is modulated by modifying the pumping power, e.g. of the laser radiation source.

21. The apparatus as set forth in claim 13, wherein said device for delivering optical radiation and forming spatial distribution of optical radiation power density on the surface and in the bulk of the biological tissue includes, optically coupled, a forming optical system and an electro-optical scanner.

22. The apparatus as set forth in claim 13, wherein said device for delivering optical radiation and forming spatial distribution of optical radiation power density on the surface and in the bulk of the biological tissue includes, optically coupled, a forming optical system and a raster system.

23. The apparatus as set forth in claim 21, wherein said forming optical system comprises a length of optical fiber, or a lens-and-mirror system adapted to deliver the laser radiation from the optical radiation source to the biological tissue area.

24. The apparatus as set forth in claim 16, wherein said information visualization and display device includes e.g. an endoscope and a display for displaying the biological tissue area, or an optical coherent tomograph.

25. The apparatus as set forth in claim 16, wherein said information visualization and display system measures geometrical characteristics of the biological tissue area.

26. The apparatus as set forth in claim 16, wherein feedback is provided by said control-diagnostic system on the basis of opto-thermal response of the biological tissue to the time-modulated laser radiation.

27. The apparatus as set forth in claim 13, wherein said feedback is provided by the control-diagnostic system on the basis of analysis of spectral content of the biological tissue response to the modulated laser radiation.

28. The apparatus as set forth in claim 13, wherein feedback is provided by the control-diagnostic system on the basis of the analysis of a amplitude and a phase of the biological tissue response to the modulated laser radiation.

29. The apparatus as set forth in claim 13, wherein the time law of the laser radiation modulation, in particular, a modulation amplitude, depth, frequency and shape are determined by the control-diagnostic system from preoperative examination data and updated during the laser treatment responsive to a control signal from the control-diagnostic system.

30. The apparatus as set forth in claim 13, wherein the formation law of the laser radiation spatial distribution is determined from preoperative examination data and updated during the laser treatment responsive to the control signal from the control-diagnostic system.

31. The apparatus as set forth in claim 13, wherein parameters of laser radiation scanning are determined from preoperative examination data and updated during the laser treatment responsive to the control signal from the control-diagnostic system.

32. The apparatus as set forth in claim 13, wherein the laser radiation modulation and spatial formation laws are coordinated on the basis of preoperative examination data and updated during the laser treatment responsive to the control signal from the control-diagnostic system.

33. The apparatus as set forth in claim 13, wherein a feedback is provided on the basis of a opto-acoustic response of the biological tissue to the modulated laser radiation formed with a predetermined spatial distribution on the surface and in the bulk of the biological tissue.

34. The apparatus as set forth in claim 13, wherein the feedback is provided on the basis of opto-electrical response of the biological tissue to the modulated laser radiation formed in accordance with a predetermined spatial distribution on the surface and in the bulk of the biological tissue.

35. The apparatus as set forth in claim 13, wherein the feedback is provided on the basis of monitoring of modification of biological tissue optical characteristics under exposure to the laser radiation modulated and formed with a predetermined spatial distribution on the surface and in the bulk of the biological tissue.

36. The apparatus as set forth in claim 16, wherein said at least one biological tissue state sensor of the control-diagnostic system is positioned directly in the biological tissue area with the aid of a surgical instrument.
